(19) United States
(12) Patent Application Publication
(10) Pub. No.: US 2021/0039087 A1
(43) Pub. Date: Feb. 11, 2021

(54) DEVICE AND METHOD FOR STORING NUCLEIC ACIDS

(71) Applicant: QIAGEN HEALTHCARE BIOTECHNOLOGIES SYSTEMS LIMITED, North Manchester (GB)

(72) Inventors: Christopher George NOREY, South Wales (GB); Kathryn Louise LAMERTON, South Wales (GB)

(21) Appl. No.: 16/652,890

(22) PCT Filed: Oct. 3, 2018
(86) PCT No.: PCT/EP2018/076932

§ 371 Date: Apr. 1, 2020

(30) Foreign Application Priority Data
Oct. 3, 2017 (GB) 1716135.7

(51) Int. Cl. B01L 3/00; C12Q 1/6806

(52) U.S. Cl. CPC B01L 3/5023; C12Q 1/6806; B01L 2300/0829; B01L 2300/044; B01L 2300/069; B01L 2200/0689

(57) ABSTRACT

Disclosed is a nucleic acids storage device comprising one or more sealable storage wells, the or each well containing one or more three dimensional solid supports capable of absorbing 5 μL or more of liquids containing any nucleic acids to be stored. Disclosed also is a method for storing nucleic acids, the method comprising, in any suitable order, the steps of: providing the device mentioned above; adding liquids, including any nucleic acids to be stored, to the storage well and thereby to be absorbed by the or each solid support in the storage well; allowing said liquids to dry substantially; sealing the or each storage well; and storing the device at room temperature.

### TECHNICAL FIELD

The present invention relates to devices and methods for the improved storage and processing of nucleic acids, such as DNA or RNA, held on solid supports such as treated cellulous fibre materials.

### BACKGROUND

Nucleic acids, such as deoxyribonucleic acids (DNA) or ribonucleic acids (RNA), have become of increasing interest as analytes for clinical or forensic uses. Powerful new molecular biology technologies enable one to detect for congenital diseases or infectious diseases. These same technologies can characterize DNA for use in settling factual issues in legal proceedings such as paternity suits and criminal prosecutions. Nucleic acid testing has been made possible due to powerful amplification methods. One can take small amounts of nucleic acids which, in and of themselves would be undetectable, and increase or amplify the amount to a degree where useful amounts are present for detection.

The most commonly employed amplification technique is known as a polymerase chain reaction, (PCR). Nucleic acid polymerases are used with template DNA from the sample in a cycled manner to create greater amounts of starting nucleic acid materials, which are easily detected, for example by electrophoresis techniques.

These known amplification techniques often provide a deliberate surplus of nucleic acids which are usually kept in cold conditions for preservation and for later possible use. Such is the scale of operations, particularly in the forensics field, that the amount of cold storage required and the energy needed to run that storage, has become a significant cost. Another option is to store surplus nucleic acids dried and at room temperature, stored on paper treated with preserving chemicals which do not significantly degrade the nucleic acids. Such papers are sold under the brand name of FTA, sold by Whatman Inc.

Such a treated paper is disclosed in U.S. Pat. No. 5,496,562 to Leigh A. Burgoyne, where an absorbent cellulose based matrix is treated with a combination of a weak base, a chelating agent, an anionic detergent, and, optionally, uric acid. The resulting product has an alkaline pH. DNA binds to this matrix and is protected against degradation.

One problem with the above mentioned paper storage is that the storage space needed is significant. For example, to prevent cross contamination between papers, the stored samples are often held in envelopes spaced from adjacent envelopes, which increases the volume of storage significantly. Another drawback is the need to manually handle stored samples or use complicated bespoke mechanisms to automate handling. Where automated handling is contemplated, the papers have a supporting card frame around them to keep them straight. The frames and/or envelopes and spaced storage means that the density of stored sample is low.

Another drawback with the above mentioned paper is that, for recovery of further amplifiable nucleic acids after storage, a portion or portions of the sample holding paper is/are removed, typically using a hollow punch, and then a number of wash, elution and amplification steps are needed. The punching step is a two step process—1) punch cleaning and 2) punching a portion usually of about 2 or 3 mm in diameter. Both steps are potential sources of cross contamination, although in practice the risk is insignificant, provided the cleaning is carried out correctly. Nevertheless, cleaning and punching take time, which slows down an automated process.

Once punched, the portion of paper can be processed according to known multi-step techniques to recover nucleic acids after said storage. However, handling of the relatively small punch paper portion(s) also requires manual intervention or bespoke handling equipment.

In place of the chemical treatment mentioned above chaotropic salts have been proposed to reduce the inhibitory burden of materials in the processing steps after punching and allow greater amounts of source DNA to be amplified, but this does not negate the practical problems of punching and sample handling after punching.

A process for isolating nucleic acids is shown in U.S. Pat. No. 5,234,809 to William R. Boom et alia, (Boom) (incorporated herein by reference). Recognizing that typical biological sources of nucleic acids can affect PCR reactions, Boom discloses using a combination of a biological source material, chaotropic salt, and a solid support, preferably finely divided glass. All three elements are combined in a liquid mixing device, with any nucleic acids present binding to the glass. After mixing, the solid support must be removed from the mixing device, washed, and the template nucleic acid eluted. Only then can it be exposed to amplification reactions.

Paper solid under the brand name FTA Elute by Whatman Inc are treated with a chaotropic salt intended to preserve nucleic acids when dried on such supports, having been deposited thereon, usually as fluid samples, for subsequent genetic characterization, primarily by conventional amplification methods such as PCR. Those supports can be used in a known protocol to collect, store, or purify nucleic acids either from a biological source, for example a biological source having naturally occurring nucleic acid amplification inhibitors present, (including either a buccal swab, cerebrospinal fluid, feces, lymphatic fluid, a plasma sample, a saliva sample, a serum sample, urine, or a suspension of cells or viruses), or from a treated whole blood biological source that has naturally occurring nucleic acid amplification inhibitors present, as well as added blood stabilization components that also inhibit nucleic acid amplification. More importantly, these nucleic acids can be released after collection or storage in a manner that enables them to be amplified by PCR. In particular, the solid supports comprise an absorbent material that does not bind nucleic acids irreversibly, and is impregnated with the chaotropic salt. A biological source sample is contacted with the impregnated absorbent material. Any nucleic acids present in the biological source can be either eluted or resolubilized off the absorbent material.

U.S. Pat. No. 6,168,922 to Michael Harvey et alia (incorporated herein by reference), describes certain embodiments of said FTA Elute and wherein it is disclosed that an absorbent material such as cellulosics, porous glasses and woven/non-woven porous polymers, can be impregnated with a chaotropic salt, to provide a releasable support for amplifiable nucleic acids, even in the presence of naturally occurring amplification inhibitors. In more detail the disclosure describes techniques to collect, store, or purify nucleic acids either from a biological source other than untreated whole blood, the biological source having naturally occurring nucleic acid amplification inhibitors present other than hemoglobin, (including samples from either a buccal swab, cerebrospinal fluid, feces, lymphatic fluid, a plasma sample, a saliva sample, a serum sample, urine, or a suspension of cells or viruses) or from a treated whole blood source that has naturally occurring nucleic acid amplification inhibitors present, as well as added blood stabilization components that also inhibit nucleic acid amplification. It is proposed that the absorbent treated material disclosed can be used to detect pathogens such as bacteria or viruses that can be found in the circulatory system. More importantly, these nucleic acids can be released after collection or storage in a manner that enables them to be amplified by conventional techniques such as PCR either by elution or re-solubilisation off the absorbent material. The device described can collect nucleic acids not only from point sources such as humans or animals, but also can be used to collect widely disseminated sources such as fungal spores, viruses, or bacterial spores, or biological material, such as bodily fluids, present at crime scenes.

### SUMMARY OF THE INVENTION

Embodiments of the present invention addresses the concerns mentioned above. The inventors have realized that an improved storage format is needed that allows easier handling, including storage of multiple samples, and convenient recovery of nucleic acids after storage. The inventors have also realized that the chemistry mentioned above employing chaotropic salts reduces the processing steps need to recover stored nucleic acids.

According to one aspect, the present invention provides a nucleic acids storage device comprising one or more sealable storage wells, the or each well containing one or more three dimensional solid supports capable of absorbing 5 μL or more of liquids containing any nucleic acids to be stored.

In an embodiment, said one or more solid supports is a single solid support having an absorbent volume of at least 7 millimeters cubed (mm3) and preferably about 7 to 180 mm3, and more preferably about 7 to 50 mm3.

In an embodiment, said one or more solid supports comprises plural solid supports, wherein each of the plural solids supports has an absorbent volume of at least 7 millimeters cubed (mm3) and preferably about 7 to 180 mm3, and more preferably about 7 to 50 mm3.

In an embodiment, the or each solid support has a thickness in each of three dimensions which three thicknesses are about equal, or where they are not equal, one dimension at least is at least 1 mm.

In an embodiment, said one or more solid supports comprises plural solid supports, wherein, in total the plural solids supports have an absorbent volume of at least 7 millimeters cubed (mm³) and preferably about 7 to 180 mm³, and more preferably about 7 to 50 mm³.

In an embodiment the solid support is coated or sorbed with a chaotropic agent, such as one or more of n-Butanol; Ethanol; Guanidinium chloride; Guanidinium/Guanidine (i so)thiocyanate; Guanidine hydrochloride; Lithium perchlorate; Lithium acetate; Magnesium chloride; Phenol 2-propanol; Sodium (iso)thiocyanate; Sodium iodide; Sodium dodecyl sulfate; Sodium perchlorate; Potassium iodide; Thiourea; and/or Urea, or a salt or salts thereof. Other chaotropic agents could be used.

In an embodiment, the storage volume and solid support have dimensions or a complementary shape which allow the placing of the, or at least one of the solid supports into the bottom of the well, such that the solid support is in contact with the lowermost part of the bottom of the well.

In an embodiment, the or each solid support is a spherical or cylindrical shape or a polyhedral shape.

In an embodiment, the one or more storage well comprises plural storage wells formed together in an array of spatially separated wells, for example a 24, 48 or 96 well array, for example each well having a closed bottom and a top opening formed in a common supporting plate.

According to a second aspect, the present invention provides a method for storing nucleic acids, the method comprising, in any suitable order, the steps of:

a) providing a storage device including plural storage wells each containing at least one absorbent solid support;
b) adding liquids, including any nucleic acids to be stored, to one or more of the storage wells and thereby to be absorbed by a respective solid support in the storage well;
c) allowing said liquids to dry substantially, optionally at a temperature above room temperature, for example up to 80 degrees Celsius and optionally in the presence of a desiccant;
d) following step c), optionally sealing each storage well; and
e) storing the device at room temperature.

In an embodiment, the above method has further step of recovering stored nucleic acids, including the steps of:

a) optionally opening the sealed storage well or where a plurality of storage wells are provided, one or more of the storage wells;
b) optionally moving at least a portion of the contents of the, or one of the storage wells into a processing well for elution or direct amplification

In an embodiment, the above method has the further step of elution of nucleic acid for amplification, including the steps of:

a) optionally adding additional wash liquids to the storage or processing well and then discarding said wash liquids but keeping the solid support;
b) adding additional liquids to the storage or processing well;
c) heating and agitating the solid support along with the additional liquids in the storage or processing well and collecting the resultant liquids for analysis.

Additional nucleic acid recovery processing steps could be employed as disclosed in co-pending patent application CN2017/085296 filed at the Chinese State Intellectual Property Office under the rules of the PCT on 22^{nd} May 2017 in the name of General Electric Company and incorporated herein by reference.

In embodiments, storage of solid supports in separated in individual wells helps to prevent cross contamination of e.g., forensic samples. This is advantageous over the current procedures whereby papers or cards need to be stored individually in pouches to prevent cross contamination, then processed by removing a small disc or punch from each card using a punching device prior to processing. This process is cumbersome, time consuming and poses a greater risk of cross-contamination. This multiplexed format is suitable for storage of forensic crime scene purified DNA samples at room temperature.

The invention extends to any features described herein. Where features are mentioned in combination herein, a claim which includes just one or a subset of said combined features is expressly considered to fall within the ambit of the invention disclosed herein.

More advantages and benefits of the present invention will become readily apparent to the person skilled in the art in view of the detailed description below.

### BRIEF DESCRIPTION OF THE DRAWINGS

The invention will now be described in more detail with reference to the appended drawings, wherein:

FIGS. 1a 1b and 1c show schematic representations of a solid support and storage well for use with the invention;

FIG. 2 shows a storage well array for use with the invention;

FIGS. 3a, 3b, 3c and 3d show different configurations of solid supports for use with the invention; and

FIGS. 4a and b show graphs of DNA yield for different experimental configurations.

### DESCRIPTION

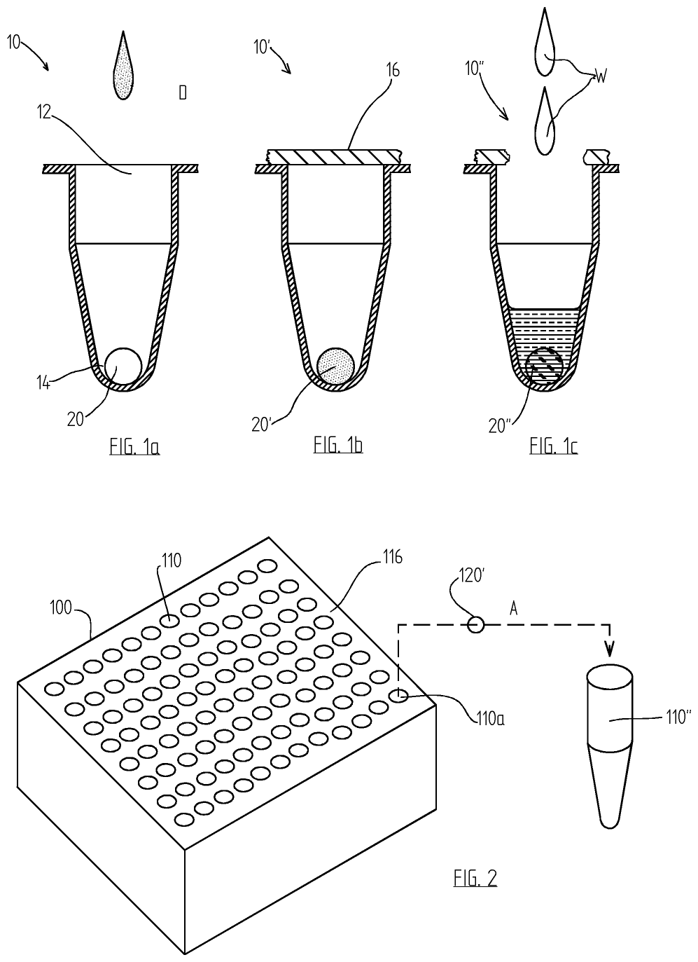

FIG. 1a shows a storage well 10, containing a spherical solid support 20, in this instance a ball of cellulose fibers of about 3.5 mm in diameter that has been dipped in a weak solution of guanidinium isothiocyanate for example containing from about 0.1 M to 6.0 M concentrations, preferably 0.5 M to 2.0 M. The absorbent material is then allowed to dry. The well has an open upper end 12 which tapers towards a rounded bottom end 14. Previously amplified DNA suspended in a liquid sample drop D is dropped into the well 10, and is absorbed by the solid support 20, then allowed to dry. The ideal amount of liquid D is enough to saturate the solid support, but not enough to allow free liquid around the solid support. The rounded bottom of the well prevents any isolated pools of liquid D remining unabsorbed by the ball 20.

FIG. 1b, shows a sealing film 16 heat sealed over the well 10′ which contains the now dried solid support 20′. Prior to sealing, whilst air drying at room temperature is possible, drying speed can be increased using elevated temperatures, for example up to 80 degrees Celsius, and/or using a desiccant material in a sealed container. The sealing film can be an impermeable barrier such as a metalized polymeric thermoplastic heat sealable film, or a similar semi-permeable film which allows water vapor out but prevents any return. Alternatively, a snap-on lid or the like could be used.

Another alternative is to use a pouch enclosing the well (or plural wells), instead of a seal/lid 16. At this stage the storage well 10′ can be stored indefinitely at room temperature without the risk is significant degradation of any nucleic acids on the ball 20′.

FIG. 1c shows the reopened well 10″ with buffer liquid W added to the well in order to recover the nucleic acid, for example by elution facilitated by washing, heating and agitation all according to the aforementioned methods described in CN2017/085296.

For simplicity, the ball 20″ is shown in the storage well 10″ in FIG. 1c. However, in practice the wells 10 and 10′ are likely to be just one of an array 100 of storage wells 110 as shown in FIG. 2. In FIG. 2, each well 110 will have a cross section as shown in FIGS. 1a, b and c, where the solid support, solid support 120′ in this embodiment, is spherical and sits snugly in the bottom of the well in contact with the well bottom. The multiple wells 110, i.e. 96 wells in number in the embodiment illustrated in FIG. 2, are likely to be used for storage only, it is most likely that the storage solid supports 120′ stored therein will be transferred, represented by arrow A, to a further processing well 110″ because the remining balls 120′ stored in the storage wells 110 of the array 100 can then be kept sealed by a common sealing film 116 and undisturbed by the usual heat and agitation used to recover the stored nucleic acids. Thus, local rupturing of the sealing film 116 at the top of one well 110a of the array 100 will allow the local ball 120′ only to be transferred to a processing well the remaining wells 110 to remain sealed, so the risk of cross contamination is eliminated.

Solid supports can be transferred manually, or by automatic means, for example using a stake to pierce the ball and move it, or without contact for example by using a nozzle emitting a gentle flow of clean air which when in close proximity to said ball 20′/120′ accelerates sufficiently to reduce pressure below atmospheric pressure and therefore allow the ball to be held in the close proximity but not touch the nozzle. Electrostatic attraction is another alternative means for lifting a solid support. Where wells 110 are removeable from the remaining array 100, there will be no need to handle the solid supports, but rather the individual well can be handled instead.

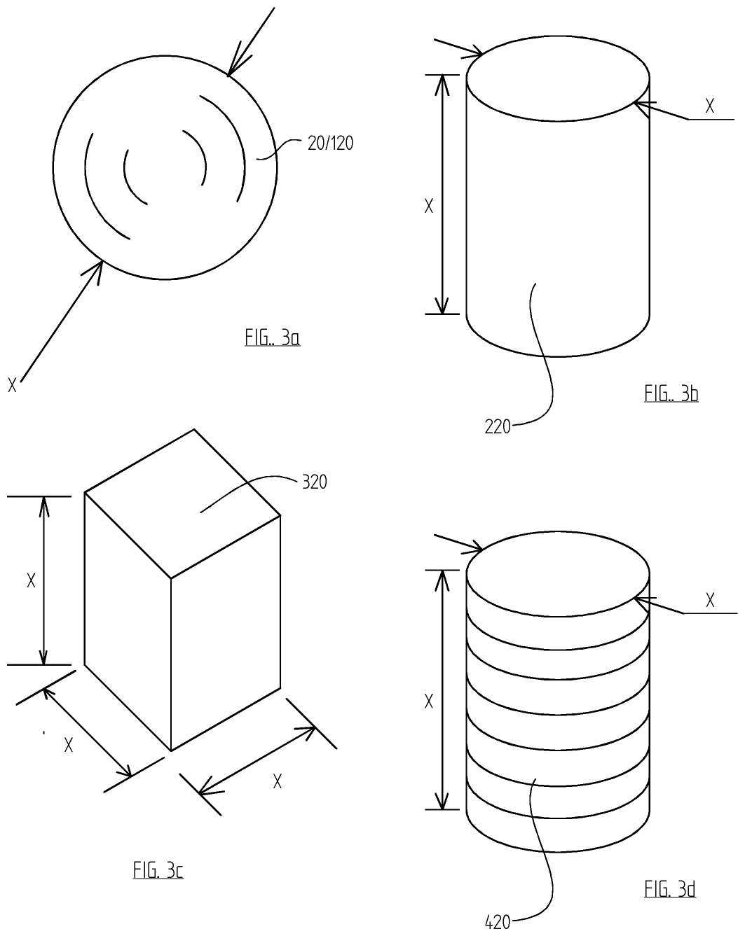

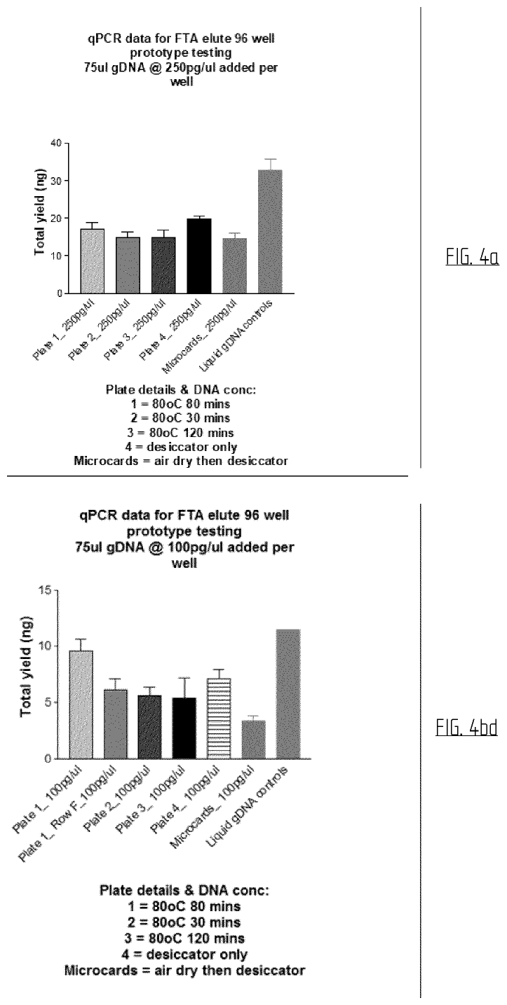

The spherical solid supports 20/120 if used singly should have a diameter of about 3.5 mm (FIG. 3a), to give a total volume of about 22 mm cubed, but other shapes and sizes could suitably be used. For example, cylindrical solid supports 220 FIG. 3b could be used, or square solid supports 320 FIG. 3c could be used. FIG. 3d shows multiple disks of sheet material, stacked to form a stack cylinder 420 equivalent in size to the cylinder 220. For uniform drying of the liquids D, the solid supports should have generally equal dimensions, such that their diameter, height, length, and width of the shapes, as denoted in the FIGS. 3a, b, c and d as dimension X are about equal. For practical reasons like strength and easy of handling, a minimum dimension, of about 1 mm is desirable, in which case it is likely that the other dimensions would be greater than 1 mm in order to obtain an absorbent volume of at least 3 mm³. However, where multiple solid supports are used, the dimension X can be as small as 1 mm. It is preferred that the solid supports make contact with the bottom of any storage well so that any liquids in the bottom of the well can be readily absorbed into the solid support. Thus, cylindrical and flat edged solid supports are more likely to be used in flat bottomed wells, for example 12, 24 or 48 well arrays which can be made flat bottomed more easily and yet still conform to the Society for Biomolecular Screening (SBS) standard outer dimensions for the arrays. 384 and 1536 SBS standard well arrays can be used with smaller size solid supports.

The material the solid supports is preferably fibrous and liquid porous in nature. Many materials are suitable for use. The main characteristics needed for the solid support material are that it is or can be made hydrophilic, and does not substantially bind nucleic acids irreversibly through either hydrophobic, ionic, covalent, or electrostatic means. The matrix must not by itself inhibit or bind amplification reactants, release substances that effect amplification reactants or otherwise affect PCR and other amplification reactions. Suitable materials include cellulosics, woven porous polymers, or non-woven porous polymers, including polyesters and polypropylenes. Cellulose fiber materials can be used, for example cellulose acetate fibers made from bleached cotton or wood pulp esterified with acetic acid. Other polymers could be used or glass fibers could be used. Some degree of absorption is preferred, but for larger wells, the solid supports can be made bigger and so just their surfaces could be made absorptive. Thus solid supports with hollow or non-absorptive cores could be used. For example a plastics polymer core could be used having a fibrous outer layer spun around it, or the polymer could be mechanically or chemically treated such that its outer surfaces have a porous or semi porous quality.

### Example 1

### Materials:

iFTAe micro cards, GE Healthcare catalogue no WB120412/WB120411
Ambion nulcease-free water (Lot—1408160)
Invitrogen Ultrapure 0.5 M EDTA, pH 8.0, catalogue no 15575-038 #1852916
Gibco 1 M TRIS, pH 8.0, catalogue no 15568-025 #1849607
Purified gDNA @50 ng/ul

### DNA IQ Spin Baskets (V1225 Promega)

1 g sachets of Desiccant, GE Healthcare WB100003
Multibarrier pouches, GE HealthcareHC WB100037
Technipaq foil pouches (for AA storage).

### Quantifiler Human DNA Quantification Kit, Applied Biosystems no 4343895 #1703209

### Preparations:

Preparation of TE buffer: (10 mM Tris, 1 mM EDTA, pH 8.0): 1 ml 1 M Tris; 200 ul 0.5 M EDTA; 98.8 ml nuclease free water=100 ml final volume.
Preparation of TE-4 buffer: (10 mM Tris, 0.1 mM EDTA, pH 8.0): 1 ml 1 M Tris; 20 ul 0.5 M EDTA; 98.98 ml nuclease free water=100 ml final volume
Preparation of DNA dilutions: 20 ng/ul, add 1.2 ml 50 ng/ul stock solution to 1.8 ml TE buffer; 2 ng/ul, add 250 ul solution 1 to 2.25 ml TE buffer.

### Control Experiment:

For the 2 ng/ul gDNA solution, 25 ul was spotted onto multiple iFTAe cards from GE Healthcare. These cards were allowed to dry in the biosafety cabinet at room temperature for 2-3 hours. Once completely dried, the cards were stored in zip lock bags with 1 g desiccant (in desiccator cabinet) at room temperature until further use.

### Main Experiment:

FTA elute cards used: #9795867
2 ng/ul gDNA solution

4×3 mm diameter disk were cut from uncontaminated FTA elute cards and each was supported on a sterile needle to form a continuous stack of punches forming a cylindrical shape. Six stacks were formed, 15 ul gDNA solution @2 ng/ul was spotted onto each stack of 4 discs (note: volume was optimised previously using sterile water):

Stack 1)—Three of the stacks were each spotted with 15 ul gDNA and placed inside a 1.5 ml Eppendorf tube to dry; and
Stack 2)—three of the stacks were spotted with 15 ul gDNA and placed upside down in Eppendorf rack to air dry.

In addition, using the control experiment, a further FTAe micro-card was spotted with 25 ul gDNA (@2 ng/ul) & dried. Then 3 mm diameter disks were cut and formed into further stacks on sterile needles, making:

Stack 3)—Three control stacks which were formed from cards which were already charged with gDNA resulting from the control experiment mentioned above. Samples were left to dry in the biosafety cabinet overnight. The following day, all samples were stored in a desiccator cabinet until required for testing.
Elution of DNA from Control Cards and Stacks

Stacks 1, 2 and 3 were then processed as follows

a) Place each stack into the bottom of a 1.5 mL microcentrifuge tube
b) Pipette 500 μL of TE-4 buffer into the microcentrifuge tube containing the 3 mm punches.
c) Close the tube and vortex the microcentrifuge tube for 5 sec.
d) Pipette off excess TE-4 buffer and discard.
e) Repeat steps 3-5 (for a total of three washes with TE-4 buffer).
f) Pipette 150 ul of TE-4 buffer into the microcentrifuge tube containing the sample.
g) Place the microcentrifuge tube on a heated mixer/shaker at 95° C. for 30 min at 1,000 rpm.
h) After incubation, briefly centrifuge the microcentrifuge tube to remove any excess liquid from the cap.
i) Place a clean spin basket into a new microcentrifuge tube.
j) Transfer the punches and eluate to the spin basket and spin at maximum speed (13 k rpm in Heraeus biofuge) for 2 min.
j) Remove the spin basket, discard the punches, and proceed with quantification and/or amplification.
l) Extracts were stored at +4° C., then quantified using a Quantifiler Human DNA Quantification Kit as per manufacturer's instructions.

### Results

| | | | | | Average yield | |
|---|---|---|---|---|---|---|
| | | | | | (ng per 150 ul = | |
| | Sample | | Quantity | Total yield | ng per 4 × 3 | % |
| Sample details | ID | Ct | (ng/ul) | (ng/150 ul) | mm punches | recovery |
| 1. FTA ball, spotted with 15 ul | 14.1A | 31.60 | 0.0838 | 12.57 | | |
| gDNA: dried in eppendorf | 14.1B | 31.37 | 0.0975 | 14.62 | 13.59 | 45.31 |
| 1 disc missing from sample 14.1C | 14.1C | 32.22 | 0.0514 | 7.71 | 11.63 | 38.77 |
| 2. FTA ball, spotted with 15 ul | 14.2A | 31.14 | 0.1137 | 17.05 | | |
| gDNA: air dried | 14.2B | 31.42 | 0.0940 | 14.11 | 16.23 | 54.11 |
| | 14.2C | 31.10 | 0.1169 | 17.54 | | |
| 3. Control, 25 ul applied to card, | 14.3A | 31.57 | 0.0853 | 12.79 | | |
| dried & 4 × 3 mm punches taken | 14.3B | 31.78 | 0.0740 | 11.10 | 10.45 | 58.03 |
| | 14.3C | 32.38 | 0.0496 | 7.44 | | |

Lines 1, 2 and 3 above represent stacks 1, 2 and 3 respectively. It should be noted that for stacks 1 and 2, the quantity DNA added to the stacks was 30 ng, whereas for stack 3 the amount was 18 ng. Therefore the percentage yield (last column) reflects this starting amount of DNA. The results demonstrate that acceptable yields of DNA can be had from a three dimensional volume of solid support, in this case a stack of paper solid supports, even if the stack is left in a well to dry.

The skilled person will appreciate that the present invention can incorporate any combination of the preferred features described above. All publications or unpublished patent applications mentioned herein are hereby incorporated by reference thereto. Other embodiments of the present invention are not presented here which are obvious to those of ordinary skill in the art, now or during the term of any patent issuing from this patent specification, and thus, are within the spirit and scope of the present invention. The invention is not to be seen as limited by the embodiments described above, but can be varied within the scope of the appended claims as is readily apparent to the person skilled in the art, for example, indefinite storage can be maintained in dry conditions, for example, by storing the sample-containing solid support in a sealed container optionally along with desiccant material, for example incorporated into the sealing film 16/116.

### Example 2

### 96 Well Testing.

### Description

Testing was undertaken of SBS standard 96 well polypropylene plates filled with a stack of 7 or 8×6 mm diameter FTA Elute discs. The plate was covered with foil containing a 5 mm diameter hole above each well.

### Sample Details:—

4×96 well plates were prepared by punching either 7 or 8×6 mm FTA Elute discs and placing them into the wells of the plate in a stack with well coordinates: D5 to D8, E5 to E8 and H1. For plate 1, discs were also placed into wells F5 to F8.
75 ul TE-4 buffer was dispensed into wells H1 (containing discs) and also empty wells H2 to H12.
75 ul gDNA at 250 pg/ul was dispensed into wells in row D
75 ul gDNA at 100 pg/ul was dispensed into wells in row E
75 ul of each gDNA solution was also dispensed onto commercially available FTA Elute microcards as a control.
Plates & microcards were dried as indicated below. Note that plates were dried with foil cover in place but no additional lid was used.

| | | Sample |
|---|---|---|
| Sample details | Storage details | Name |
| Plate 1_row D 250 pg/ul | 80° C. for 80 mins, then placed in | 1 |
| (8 discs per well) | desiccator drying cabinet | |
| Plate 2_row D 250 pg/ul | 80° C. for 30 mins then placed in | 3 |
| (7 discs per well) | desiccator drying cabinet | |
| Plate 3_row D 250 pg/ul | 80° C. for 120 mins, then placed in | 5 |
| (7 discs per well) | desiccator drying cabinet | |
| Plate 4_row D 250 pg/ul | Placed directly in desiccator | 7 |
| (7 discs per well) | drying cabinet | |
| Microcards_250 pg/ul (*) | Air dried at room temperature, | 13 |
| | then placed in desiccator cabinet | |
| Plate 1_Row E_100 pg/ul | 80° C. for 80 mins, then placed in | 2 |
| (8 discs per well) | desiccator drying cabinet | |
| Plate 1_Row F_100 pg/ul | 80° C. for 80 mins, then placed in | 16 |
| (8 discs per well) | desiccator drying cabinet | |
| Plate 2_Row E 100 pg/ul | 80° C. for 30 mins, then placed in | 4 |
| (7 discs per well) | desiccator drying cabinet | |
| Plate 3_Row E 100 pg/ul | 80° C._120 mins, then placed in | 6 |
| (7 discs per well) | desiccator drying cabinet | |
| Plate 4_Row E 100 pg/ul | Placed directly in desiccator | 8 |
| (7 discs per well) | drying cabinet | |
| Microcards_100 pg/ul | Air dried, then placed in | 14 |
| | desiccator drying cabinet | |

### Materials:

1. iFTA Elute microcards, code WB120411 #9852980 Exp May 2020
2. gDNA, Promega DD7251 #0000286658 Exp 3 Apr. 2020 @250 pg/ul
3. Quantifiler human DNA Quantification Kit, Applied Biosystems cat 4343895 #1712213, exp 19 Jun. 2019
4. DNA IQ spin baskets, Promega V1225 #0000232618
5. Millipore Microcon DNA Fast Flow devices, MCF0R100 #R7BA83255
6. Promega Powerplex Fusion Kit, DC2402 #0000293543 Exp 20 Jan. 2019

### Equipment

Pipettes
   P10, CL/IN/PI/00092
   P100, CL/IN/PI/00110
   P1000, CL/IN/PI/00128
   Multipette, Serial G14946G

All pipettes—calibration due end September 2018
7900 real time PCR machine, Applied Biosystems, CL/LE/PE/00293, next calibration due August 2018
9700 thermal Cycler, CL/LE/AE/00656, calibration not required.
3500×1 genetic analyser, Thermo Fisher: CL/LE/PE/000489. Due for service end July 2018

### Method

1. Preparation of TE-1 buffer (10 mM Tris, 1 mM EDTA, pH 8.0), comprising:

### 1 ml of 1 M Tris

### 200 ul of 0.5 M EDTA

98.8 ml nuclease free water
100 ml final volume

2. Preparation of TE-4 buffer (10 mM Tris, 0.1 mM EDTA, pH 8.0), comprising:

### 1 ml of 1 M Tris

### 20 ul of 0.5 M EDTA

98.98 ml nuclease free water
100 ml final volume

3. All FTAe microcards were spotted, punched and processed as below:—

### Methodology

1. For microcards: 7×6 mm diameter punch samples were taken from each card.
2. For discs in 96 well plates, each ‘stack’ of 7/8 dried discs were ‘spiked’ with a sterile needle, removed from the well & transferred to a 2 ml sterile eppendorf tube prior to processing as for the microcards (proceed directly to section 3 below).
3. Measure DNA yield from samples using Quantifiler Human DNA Quantification kit using an ABI™ 7900HT Fast Real-Time PCR System.
   Elution of gDNA from FTA Elute Microcards: Adapted from FTA Elute Procedure 29250657AA
   1. Place the FTA elute microcard on a cutting mat.
   2. Remove seven, 6 mm punches from the FTA Elute Card and place the punches into a single 2.0 mL microcentrifuge tube.
   3. Pipette 1000 μL of TE-4 buffer into the microcentrifuge tube containing the 6 mm punches.
   4. Close the tube and vortex the microcentrifuge tube for 5 seconds. Ensure the punches move up into the centre of the microcentrifuge tube when they are vortexed.

NOTE: If the punches remain at the bottom of the microcentrifuge tube during vortexing, they will not be washed adequately.

5. Pipette off excess TE-4 buffer and discard.

Note: Remove ALL excess buffer between wash steps.

6. Repeat steps 3-5 (for a total of three washes with TE-4 buffer).
7. Pipette 400 ul of TE-4 buffer into the microcentrifuge tube containing the sample punches.
8. Place the microcentrifuge tube on a heated mixer/shaker at 95° C. for 30 min at 1,000 rpm.
9. After incubation, briefly centrifuge the microcentrifuge tube to remove any excess liquid from the cap.
10. Place a clean spin basket into a new microcentrifuge tube. Transfer the punches and eluate to the spin basket and spin at maximum speed for 2 min.
11. Remove the spin basket, discard the punches, and proceed with quantification and/or amplification.

NOTE: If the sample is too dilute to meet the DNA input needed for PCR amplification, the sample can be concentrated.

Concentrate any DNA extracts <0.033 ng/ul (equivalent to 0.5 ng/15 ul) using Millipore Microcon DNA Fast Flow devices.
Measuring DNA Yield from Samples Using Quantifiler Human DNA Quantification Kit Using an ABI™ 7900HT Fast Real-Time PCR System.

2 ul DNA extracts were added to 23 ul of the following reaction mix:—

### Master Mix Preparation for 100 Reactions:—

### Qfiler Human Primer Mix: 1050 ul

Qfiler PCR reaction mix: 1250 ul
Total volume: 2300 ul
Add 23 ul per well

### Thermal Cycling Protocol on AB 7900 Real Time PCR Instrument:—

95° C. for 10 mins, then:
95° C. for 15 secs
60° C. for 60 secs
For 40 cycles

Standard curve prepared as per Manufacturer's instructions.

Note: samples of diluted gDNA (i.e., solutions that were spotted onto cards) were included in the qPCR.

Following qPCR analysis, all samples spiked with gDNA at 100 pg/ul were concentrated as described below:—

### Concentration of DNA Extracts Using Millipore Microcon DNA Fast Flow.

Concentrate any DNA extracts where concentration was <0.033 ng/ul using Millipore Microcon DNA Fast Flow devices.

Note: 0.033 ng/ul is equivalent to 0.5 ng/15 ul. The Powerplex Fusion kit allows 15 ul sample addition, minimum quantity of DNA is 0.5 ng.

### How to Use the Microcon® Filter Device:

NOTE: For Microcon® DNA Fast Flow PCR Grade devices, use aseptic technique when opening packages and throughout the procedure. Carefully reseal pouches to protect unused samples from contamination.

1. Insert Microcon® device into tube.
2. Pipette solution into device (0.5 mL maximum volume), taking care not to touch the membrane with the pipette tip. Seal with attached cap.
3. Place assembly in a compatible centrifuge (described in the Equipment Required section) and counterbalance with a similar device.

NOTE: When placing the assembled device into the centrifuge rotor, align the cap strap toward the center of the rotor.

4. Spin at 500×g for DNA Fast Flow devices=2,300 rpm in Hereaus Biofuge for 20 mins.
5. Remove assembly from centrifuge. Separate tube from filter device.
6. Place a new tube over the top of the device. Invert the assembly and centrifuge for 3 minutes at 1,000×g (or pulse briefly) to transfer concentrate to tube=3,200 rpm in Hereaus Biofuge.
7. Remove from centrifuge. Separate tube from filter device. Close sealing cap to store sample for later use.

For volumes of DNA extract eluted from Microcon devices & calculations to provide 0.5 ng per PCR reaction—refer to attachment 1

### PCR Amplification of DNA Extracts Using the PowerPlex® Fusion System:

Calculate volume of DNA extract to provide 0.5 ng DNA. Make final volume of sample up to 15 ul using sterile water. Add 15 ul sample to appropriate wells of a 96 well plate:

Note:
For samples spiked with gDNA at 250 pg/ul, 15 ul eluate was used for each STR reaction.
For samples spiked with gDNA at 100 pg/ul, 3 ul of concentrated eluate was used for each STR reaction.

For details of actual qty of gDNA added per STR reaction, refer to attached Excel spreadsheet containing qPCR data.

### Master Mix Preparation for 100 Reactions:—

### Fusion 5× Master Mix: 500 ul

### Fusion 5× Primer mix: 500 ul

Total volume: 1000 ul
Add 10 ul per well
(plus 15 ul sample=25 ul per well)

### Thermal Cycling Protocol, 9700 Thermal Cycler:—

96° C. for 1 minute, then:
94° C. for 10 secs

### 59° C. for 1 min

72° C. for 30 secs
For 30 cycles, then:
60° C. for 10 minutes
4° C. soak

### Preparation of Amplified Samples for STR Analysis (100 Reactions):—

### WEN ILS: 50 ul

### Hi Di Formamide: 950 ul

Total volume: 10 ml
Add 10 ul per well

Note: control DNA was added to appropriate wells at 1 ng/ul, 100 pg/ul and 20 pg/ul.

### Results

Summary of qPCR Data:—

Samples spiked with gDNA at 250 pg/ul

| | | | | | % recovery | % recovery |
|---|---|---|---|---|---|---|
| | | | Average | % recovery | (compared to liquid | (compared to |
| | | | total | compared to | gDNA control - | Promega stated |
| | | Sample | yield | microcard | solution spotted | DNA concentra- |
| Sample details | Storage details | Name | (ng) | control | onto cards) (**) | tion) (***) |
| Plate 1_250 pg/ul | 80° C._80 mins, | 1 | 17.09 | 118.03 | 52.38 | 91.13 |
| (8 discs per well) | desiccator | | | | | |
| Plate 2_250 pg/ul | 80° C._30 mins, | 3 | 14.86 | 102.66 | 45.56 | 79.27 |
| (7 discs per well) | desiccator | | | | | |
| Plate 3_250 pg/ul | 80° C._120 mins, | 5 | 14.71 | 101.61 | 45.09 | 78.45 |
| (7 discs per well) | desiccator | | | | | |
| Plate 4_250 pg/ul | Desiccator only | 7 | 19.86 | 137.21 | 60.89 | 105.94 |
| (7 discs per well) | | | | | | |
| Microcards_250 pg/ul (*) | Air dried then | 13 | 14.48 | | 44.38 | 77.21 |
| | desiccator | | | | | |
| CONTROL: liquid gDNA | | | 32.62 | | | |
| (used to spike FTAe) | | | | | | |

| | |
|---|---|
| Yield calculate from qPCR data for | 32.62 ng |
| liquid gDNA (used to spike FTAe) (**) | |
| Yield calculated from Promega's stated concentration | 18.75 ng |
| of 250 pg/ul (250 pg × 75 ul) = 18.75 ng (***) | |

Samples spiked with gDNA at 100 pg/ul

| | | | | | % recovery | % recovery |
|---|---|---|---|---|---|---|
| | | | Average | % recovery | (compared to liquid | (compared to |
| | | | total | compared to | gDNA control - | Promega stated |
| | | Sample | yield | microcard | solution spotted | DNA concentra- |
| Sample details | Storage details | Name | (ng) | control | onto cards) (**) | tion) (***) |
| Plate 1_100 pg/ul | 80° C._80 mins, | 2 | 9.55 | 254.89 | 83.22 | 127.35 |
| (8 discs per well) | desiccator | | | | | |
| Plate 1_Row F_100 pg/ul | 80° C._80 mins, | 16 | 6.16 | 164.31 | 53.65 | 82.09 |
| (8 discs per well) | desiccator | | | | | |
| Plate 2_100 pg/ul | 80° C._30 mins, | 4 | 5.61 | 149.64 | 48.86 | 74.76 |
| (7 discs per well) | desiccator | | | | | |
| Plate 3_100 pg/ul | 80° C._120 mins, | 6 | 5.41 | 144.34 | 47.13 | 72.11 |
| (7 discs per well) | desiccator | | | | | |
| Plate 4_100 pg/ul | Desiccator only | 8 | 7.10 | 189.40 | 61.84 | 94.63 |
| (7 discs per well) | | | | | | |
| Microcards_100 pg/ul (*) | Air dried then | 14 | 3.75 | | 32.65 | 49.96 |
| | desiccator | | | | | |
| CONTROL: liquid gDNA | | | 11.48 | | | |
| (used to spike FTAe) | | | | | | |

| | |
|---|---|
| Yield calculated from qPCR data for | 11.48 ng |
| liquid gDNA (used to spike FTAe) (**) | |
| Yield calculated from Promega's stated concentration | 7.5 ng |
| of 250 pg/ul (250 pg × 75 ul) = 18.75 ng (***) | |

DNA yield from all 96-well prototypes samples were equivalent to, or better than (p>0.05) the microcard control (Mann Whitney non parametric t-test AND unpaired t-test with Welch's correction).

Note: in the tables above, % recovery was calculated using two methods:
1. Promega purchased gDNA was assumed to be at the concentration specified on the vial.
2. Using concentration calculated by qPCR.
3. Also, yield was calculated assuming 500 ul elution volume, however this is usually closer to ˜550 ul (Note; 400 ul TE-4 buffer is added for the elution step, but there is usually ˜150 ul residual buffer remaining on the punches—giving ˜550 ul).

Controls

| | Sample | Average total |
|---|---|---|
| Sample details | Name | yield (ng) |
| plate 2 blank punches | 10 | 0.00 |
| plate 3 blank punches | 11 | 0.00 |
| plate 4 blank punches | 12 | 0.00 |
| Microcards_Blank punches | 15 | 0.00 |

Graphs of DNA yield for different initial concentrations of DNA are illustrated in FIGS. 4a and 4b

### Summary of STR Data:—

Samples spiked with gDNA at 250 pg/ul

| | | | | | | | Ratio small:large loci (indicator for degradation if too high) | | | | |
|---|---|---|---|---|---|---|---|---|---|---|---|
| | | | | | | | | | | | Actual ng |
| | | | | | | | | | | | DNA added |
| | Storage/drying | Sample | No | % | | PHR < | | | | | to STR |
| Sample details | conditions | number | Alleles | FPY | APH | 0.5 | Blue | Green | Black | Red | reaction |
| Plate 1 | 80° C. for 80 mins, | n = 4 | 172 | 100 | 7058.8 | 0 | 2.18879 | 2.061882 | 0.922889 | 1.463144 | 1.62 |
| (rowE)_75 ul | then desiccator | | | | | | | | | | |
| @100 pg/ul | | | | | | | | | | | |
| Plate 1 | 80° C. for 80 mins, | n = 4 | 172 | 100 | 5507.6 | 0 | 2.104329 | 1.906848 | 0.767307 | 1.447637 | 1.05 |
| (row F)_75 ul | then desiccator | | | | | | | | | | |
| @ 100 pg/ul | | | | | | | | | | | |
| Plate 2_75 ul | 80° C. for 30 mins, | n = 4 | 172 | 100 | 5050.0 | 0 | 2.724707 | 2.427114 | 1.022298 | 1.599129 | 0.95 |
| @ 100 pg/ul | then desiccator | | | | | | | | | | |
| Plate 3_75 ul | 80° C. for 120 mins, | n = 4 | 172 | 100 | 6534.0 | 0 | 2.642232 | 2.378989 | 1.178983 | 2.008145 | 0.92 |
| @100 pg/ul | then desiccator | | | | | | | | | | |
| Plate 4_75 ul | desiccator only | n = 4 | 172 | 100 | 7330.6 | 0 | 2.414891 | 2.599608 | 1.240129 | 2.229328 | 1.21 |
| @100 pg/ul | | | | | | | | | | | |
| Microcards_75 ul | desiccator only | n = 3 | 129 | 100 | 3314.3 | 0 | 2.808558 | 2.557776 | 1.27328 | 2.077607 | 0.57 |
| @100 pg/ul | | | | | | | | | | | |
| Liquid sample of | | n = 2 | 86 | 100 | 8510.9 | 0 | 1.002449 | 1.167766 | 0.67528 | 0.590126 | |
| gDNA used to | | | | | | | | | | | |
| spike cards | | | | | | | | | | | |

Samples spiked with gDNA at 100 pg/ul

| | | | | | | | Ratio small:large loci (indicator for degradation if too high) | | | | |
|---|---|---|---|---|---|---|---|---|---|---|---|
| | | | | | | | | | | | Actual ng |
| | | | | | | | | | | | DNA added |
| | Storage/drying | Sample | No | % | | PHR < | | | | | to STR |
| Sample details | conditions | number | Alleles | FPY | APH | 0.5 | Blue | Green | Black | Red | reaction |
| Plate 1_75 ul | 80° C. for 80 mins, | n = 4 | 172 | 100 | 3555.4 | 0 | 2.13481 | 2.243848 | 0.995151 | 1.556645 | 0.51 |
| @250 pg/ul | then desiccator | | | | | | | | | | |
| Plate 2_75 ul | 80° C. for 30 mins, | n = 4 | 172 | 100 | 3108.5 | 0 | 2.874924 | 1.990518 | 0.959579 | 1.684184 | 0.45 |
| @ 250 pg/ul | then desiccator | | | | | | | | | | |
| Plate 3_75 ul | 80° C. for 120 mins, | n = 4 | 172 | 100 | 3718.9 | 0 | 2.646956 | 2.256263 | 1.032001 | 1.667869 | 0.44 |
| @250 pg/ul | then desiccator | | | | | | | | | | |
| Plate 4_75 ul | desiccator only | n = 4 | 172 | 100 | 3746.6 | 0 | 2.563884 | 1.989473 | 1.034681 | 1.579183 | 0.60 |
| @250 pg/ul | | | | | | | | | | | |
| Microcards_75 ul | desiccator only | n = 6 | 254 | 98.4 | 2241.3 | 3 | 2.5999 | 2.521164 | 1.343501 | 2.541144 | 0.43 |
| @250 pg/ul | | | | | | | | | | | |
| Liquid sample of | | n = 1 | 43 | 100 | 7719.4 | 0 | 1.250287 | 0.991955 | 0.739027 | 0.740281 | |
| gDNA used to | | | | | | | | | | | |
| spike cards | | | | | | | | | | | |

A 100% full pass yield was achieved for all samples tested, apart from 100 pg/ul microcard control.
All ratio's for small:large loci below 3.0, also 96-well plate ratios comparable to microcard control, therefore DNA quality comparable for plates v microcards.

Note: however ALL liquid control ratios are below 2.0

### CONCLUSIONS

DNA yield from all 96-well prototypes samples were equivalent to, or better than (p>0.05) the microcard control (Mann Whitney non parametric t-test AND unpaired t-test with Welch's correction).
STR data (indication of DNA quality):
100% FPY achieved for all samples tested, apart from 100 pg/ul microcard control which was 98.4%
All PHR's were above 0.5, apart from the microcard control
All ratio's for small:large loci were below 3.0, also 96-well plate ratios comparable to microcard control, therefore DNA quality comparable for plates v microcards.

It was found that DNA yield and quality from 96-well plate prototypes obtained bt Example 2 were comparable (if not better) than FTA Elute microcards used conventionally.

1. A nucleic acids storage device comprising one or more sealable storage wells, the or each well containing one or more three dimensional solid supports, the or each support capable of absorbing 5 μL or more of liquids containing any nucleic acids to be stored.

2. The device as claimed in claim 1, wherein the or each solid support comprises a stack of pieces or punches taken from a sheet material.

3. The device as claimed in claim 1, wherein the sum total liquids absorbable by the solid support or where more than one solid support is used all the solid supports, is at least 5 μL.

4. The device as claimed in claim 1, wherein said one or more solid supports comprises plural solid supports, wherein each of the plural solids supports has an absorbent volume of at least 7 millimeters cubed (mm3) or wherein in total the plural solids supports have an absorbent volume of at least 7 millimeters cubed (mm3).

5. The device as claimed in claim 1, wherein, said one or more solid supports is a single solid support having an absorbent volume of at least 7 millimeters cubed (mm3).

6. The device as claimed in claim 1, wherein the or each solid support has: a thickness in each of three dimensions which three thicknesses are about equal; or at least 1 mm in thickness in one dimension.

7. The device as claimed in claim 1, wherein the or each solid support includes: fibers of cellulose or other polymeric material; and/or glass fibers.

8. The device as claimed in claim 1, wherein the or each solid support is coated or sorbed with a chaotropic agent, selected from one or more of n-Butanol; Ethanol; Guanidinium chloride; Guanidinium/Guanidine (iso)thiocyanate; Guanidine hydrochloride; Lithium perchlorate; Lithium acetate; Magnesium chloride; Phenol 2-propanol; Sodium (iso)thiocyanate; Sodium iodide; Sodium dodecyl sulfate; Sodium perchlorate; Potassium iodide; Thiourea; and/or Urea, or a salt or salts thereof.

9. The device as claimed in claim 1, wherein the storage volume and solid support have dimensions or a complementary shape which allow the placing of the, or at least one of the solid supports into the bottom of the well, such that the solid support in the bottom of the well is in contact with the lowermost part of the bottom of the well.

10. The device as claimed in claim 1, wherein the or each solid support is a spherical or cylindrical shape, or a polyhedral.

11. The device as claimed in claim 1, wherein the one or more storage well comprises plural storage wells formed together in an array of spatially separated wells.

12. The device as claimed in claim 11, wherein the array includes a sealing film for sealing all the top openings.

13. A method for storing nucleic acids, the method comprising, in any suitable order, the steps of:
a) providing a storage device including plural storage wells each containing at least one absorbent solid support;
b) adding liquids, including any nucleic acids to be stored, to one or more of the storage wells and thereby to be absorbed by a respective solid support in the storage well;
c) allowing said liquids to dry substantially, optionally at a temperature above room temperature and optionally in the presence of a desiccant;
d) following step c), optionally sealing each storage well; and
e) storing the device at room temperature.

14. The method according to claim 13, comprising the further step of recovering stored nucleic acids, including the steps of:
a) optionally opening the sealed storage well or where a plurality of storage wells are provided, one or more of the storage wells;
b) optionally moving at least a portion of the contents of the, or one of the storage wells into a processing well for elution or direct amplification

15. The method according to claim 14, comprising the further step of elution of nucleic acid for amplification, including the steps of:
a) optionally adding additional wash liquids to the storage or processing well and then discarding said wash liquids but keeping the solid support;
b) adding additional liquids to the storage or processing well;
c) heating and agitating the solid support along with the additional liquids in the storage or processing well and collecting the resultant liquids for analysis.
